(19) United States
(12) Patent Application Publication
(10) Pub. No.: US 2021/0186548 A1
(43) Pub. Date: Jun. 24, 2021

(54) Handpiece Tip

(71) Applicant: Eunsung Global Corp., Wonju-si (KR)

(72) Inventor: Ki Se Lee, Seoul (KR)

(21) Appl. No.: 16/996,173

(22) Filed: Aug. 18, 2020

(30) Foreign Application Priority Data
Dec. 19, 2019 (KR) 10-2019-0171193

(51) Int. Cl. A61B 17/32

(52) U.S. Cl. CPC A61B 17/32; A61B 2017/00761; A61B 2017/320008

(57) ABSTRACT

A handpiece tip is proposed. The handpiece tip includes a contact part to contact the skin, and a coupling part having a space formed therein and coupled to a handpiece body, wherein the contact part includes a surface, a protruding part provided to protrude on an outer side of the surface and touching the skin, a discharging hole formed on the surface to discharge a therapeutic liquid, and a suctioning hole formed on the surface so as to suction a mixture of dead skin cells and the liquid. According to the disclosure, there are effects in that nutrient solution is injected by way of closely contacting the skin, the layers of dead skin cells are removed, and desired abrasive performance on the skin is realized while minimizing skin damage.

### CROSS REFERENCE TO RELATED APPLICATION

The present application claims priority to Korean Patent Application No. 10-2019-0171193, filed Dec. 19, 2019, the entire contents of which is incorporated herein for all purposes by this reference.

### BACKGROUND OF THE INVENTION

### Field of the Invention

The content disclosed herein relates to a handpiece tip and, more particularly, to a handpiece tip provided on a handpiece that removes dead skin cells of the skin.

### Description of the Related Art

Unless otherwise indicated in the present disclosure, the content described in this section is not related art to the claims of this application and is not admitted to be the related art by inclusion in this section.

In general, as for exfoliating the skin, there are provided types of devices including: a peeling device in which a manufactured skin peeling substance is applied to the skin or crystal powder is infused into suctioned air of the device, whereby the fine powder hits the skin surface and the skin is peeled off, and a peeling device (also known as a diamond peeling device) provided with a cylindrical shape perforated rod having one side thereof on which diamond powder is directly applied so that the skin may be peeled off thereby.

The crystal peeling device requires too much amount of crystal powder to peel the skin while infusing the crystal powder, and is also expensive. As for the diamond peeling device, since the device's friction surface with the skin is rough, the device easily amasses skin dirt, has severe bacterial growth, and is unsanitary. In addition, although an ultrasonic cleaner is used to clean the diamond peeling device, there are problems in that complete removal of sticky foreign matter stuck on an abrasive surface of a handpiece is difficult, and a concern of infection occurs due to the unsanitary surface that other people should continually use in turn. There is also an economic issue in that an expensive diamond tip should be discarded after using the tip several times.

Meanwhile, in Korean Utility Model Registration No. 20-0427899, “HANDPIECE HAVING DISPOSABLE ABRASIVE TIP” is disclosed.

The related art is provided with an abrasive tip having opposite sides or a single side thereof coated with crystal powder or metallic powder, which looks like sandpaper; having a rough surface of an abrasive stone; and having a coin shape in which the center thereof is perforated and a suction passage is formed in the middle.

However, in the related art, there is a concern that the abrasive fine powder may remain on the skin, which is unsanitary and may cause excessive skin damage.

### DOCUMENT OF RELATED ART

(Patent Document 1) Korean Utility Model Registration No. 20-0427899

### SUMMARY OF THE INVENTION

The objective of the present disclosure is to provide a handpiece tip capable of injecting a nutrient solution by way of closely contacting the skin, removing layers of dead skin cells, and realizing desired abrasive performance on the skin while minimizing skin damage.

The objective of exemplary embodiments is achieved by a handpiece tip including: a contact part to contact the skin; and a coupling part having a space formed therein and coupled to a handpiece body, wherein the contact part includes a surface, a protruding part provided to protrude on an outer side of the surface and touching the skin, a discharging hole formed on the surface to discharge a therapeutic liquid, and a suctioning hole formed on the surface so as to suction a mixture of dead skin cells and the liquid.

The protruding part may be provided with a curved part to have a rounded corner portion, thereby preventing skin damage.

The handpiece tip may further include: a guide wall protrudingly provided on the outer side of the surface of the contact part; and a plurality of partition walls connected to an inner circumferential surface of the guide wall and provided on the outer side of the surface of the contact part.

The handpiece tip may further include: a plurality of bits provided on the surface of the contact part and protrudingly provided in a circumferential direction around the discharging hole; and a flow path provided between the plurality of bits to allow the fluid discharged from the discharging hole to flow.

The flow path may be provided with a flow path bottom on the surface of the contact part, and the flow path bottom may be configured as a horizontal bottom or as an inclined bottom inclined toward the suctioning hole from the discharging hole.

The discharging hole may be formed on a central part of the surface of the contact part, and the suctioning hole may be formed at an outer circumferential edge of the surface of the contact part.

The surface of the contact part may be configured to be inclined downward toward the suctioning hole from the discharging hole.

The protruding part may include a plurality of walls protruding in a concentric circular shape or a spiral shape.

The flow path through which fluid flows may be provided between the plurality of walls.

The concentric circular wall or spiral wall may be provided to have a lower height than that of the guide wall.

The protruding part may include a plurality of domes configured to protrude in a hemispherical shape on the surface of the protruding part.

The plurality of domes may be arranged in the circumferential direction on the outer circumferential edge of the discharging hole.

The surface of the contact part may be configured to protrude in the hemispherical shape with the discharging hole as a peak.

According to the exemplary embodiments disclosed herein, there are the effects in that the nutrient solution is injected by way of closely contacting the skin, the layers of dead skin cells are removed, and desired abrasive performance on the skin is realized while minimizing skin damage.

### BRIEF DESCRIPTION OF THE DRAWINGS

FIG. 1 is a perspective view showing a handpiece beauty device according to an exemplary embodiment.

FIG. 2 is a perspective view showing a bit-type handpiece tip according to a first exemplary embodiment.

FIG. 3 is a cross-sectional view showing the bit-type handpiece tip according to the first exemplary embodiment.

FIG. 4 is a cross-sectional view showing another form of the handpiece tip.

FIG. 5 is a perspective view showing a handpiece tip having a protruding part having a concentric circular shape according to a second exemplary embodiment.

FIG. 6 is a perspective view of a handpiece tip having a dome-shaped protruding part according to a third exemplary embodiment.

### DETAILED DESCRIPTION OF THE INVENTION

Hereinafter, exemplary embodiments of the present disclosure will be described in detail with reference to the accompanying drawings.

The embodiments to be described below are intended to be described in detail so that those skilled in the art to which the present invention pertains may easily implement the disclosure, and thus it does not mean that the technical spirit and scope of the present disclosure are limited.

In addition, the size or shape of the components shown in the drawings may be exaggerated for clarity and convenience of description, terms specifically defined in consideration of the configuration and operation of the present disclosure will vary depending on the intention or custom of the user or operator, and it should be understood that definitions of these terms should be made based on the contents throughout the disclosure.

### First Exemplary Embodiment

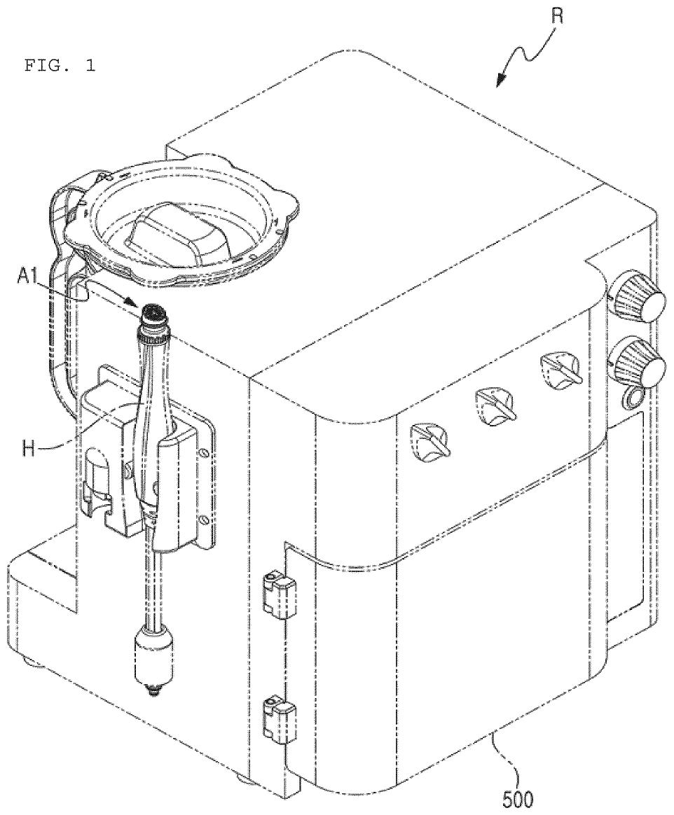

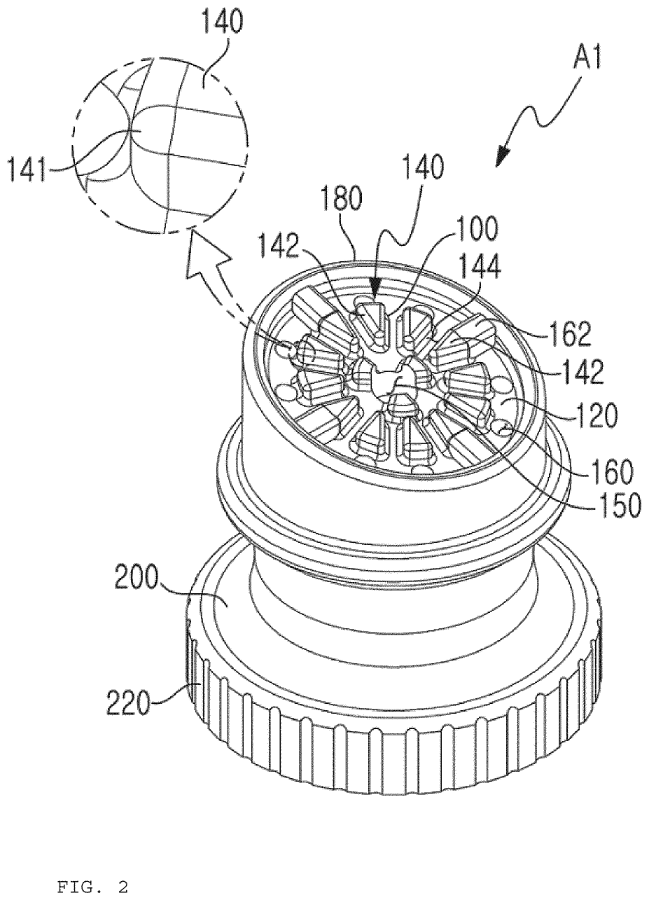

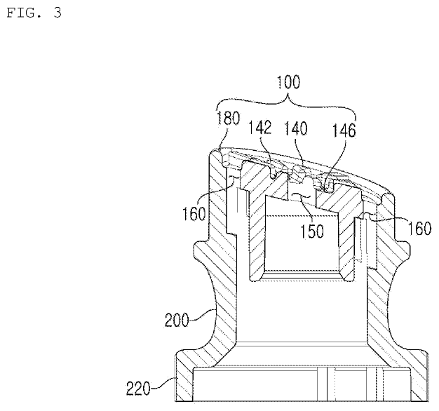

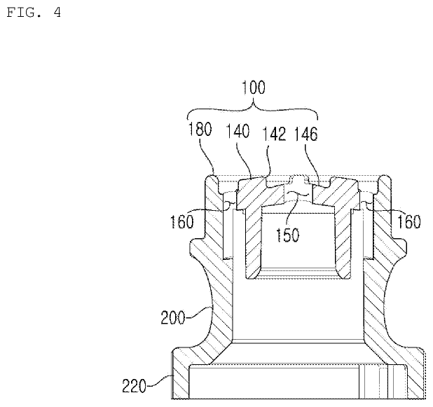

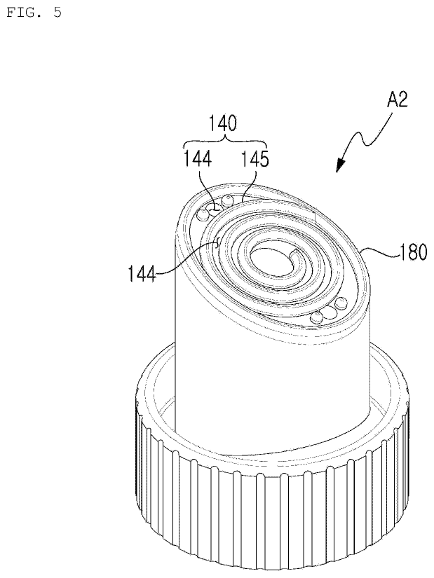

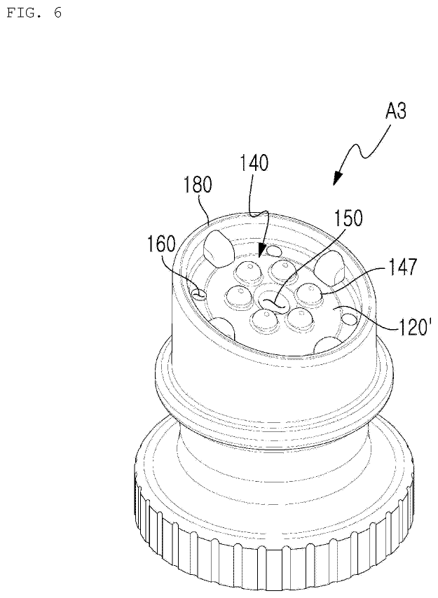

Among the accompanying views, FIG. 1 is a perspective view showing a handpiece beauty device according to an exemplary embodiment, FIG. 2 is a perspective view showing a bit-type handpiece tip according to a first exemplary embodiment, FIG. 3 is a cross-sectional view showing the bit-type handpiece tip according to the first embodiment, FIG. 4 is a cross-sectional view showing another form of the handpiece tip, FIG. 5 is a perspective view showing a handpiece tip having a protruding part having a concentric circular shape according to a second exemplary embodiment, and FIG. 6 is a perspective view showing a handpiece tip having a dome-shaped protruding part according to a third exemplary embodiment.

As shown in FIG. 1, a handpiece beauty device R according to the exemplary embodiment includes a main body 500, a handpiece body H connected to the main body 500 by a cable, and a handpiece tip A1 coupled to the handpiece body H.

The bit-type handpiece tip according to the first exemplary embodiment, as shown in FIGS. 2 to 4, includes: a contact part 100 to contact the skin; and a coupling part 200 having a space formed therein and coupled to the handpiece body H.

The contact part 100 includes: a surface 120; a protruding part 140 provided to protrude on an outer side of the surface 120 and touching the skin; a discharging hole 150 formed on the surface 120 to discharge a therapeutic liquid; and a suctioning hole 160 formed on the surface 120 so as to suction a mixture of dead skin cells and the liquid.

The protruding part 140 is provided to have a curved part 141 so that a corner portion is rounded, thereby preventing skin damage.

The coupling part 200 is knurled on the outer circumferential surface thereof to form a plurality of protrusions 220 and is provided to be fastened to the head of the handpiece body H.

The handpiece tip includes: a guide wall 180 provided to protrude on the outer side of the surface 120; and a plurality of partition walls 162 connected to the inner circumferential surface of the guide wall 180 and provided on the outer side of the surface 120.

According to the exemplary embodiment, the protruding part 140 is provided with a plurality of bits 142 provided on the surface 120 and protrudingly provided in the circumferential direction around the discharging hole 150; and a flow path 144 provided between the plurality of bits 142 to allow the fluid discharged from the discharging hole 150 to flow therethrough.

The bit 142 is protrudingly provided in an approximately triangular shape. In addition, the upper edge of the bit 142 is configured to be round.

A mixture of dead skin cells and fluid flows through the flow path 144 to be able to move to the suctioning hole 160.

As shown in FIG. 3, the flow path 144 is provided with a flow path bottom 146 on the surface 120, wherein the flow path bottom 146 is configured as a horizontal bottom, but is provided in an inclined shape slanted to one side thereof.

Therefore, the fluid moves along on the flow path bottom 146, but is transferred in an inclined direction.

Alternatively, as shown in FIG. 4, according to another form, the flow path bottom 146 may be configured to be inclined downward toward the suctioning hole 160 with the discharging hole 150 as a peak.

According to the exemplary embodiment, the surface 120 having a central part thereof is provided with the discharging hole 150 therein, and is provided with the suctioning hole 160 on the outer circumferential edge of the surface 120.

The surface 120 is provided to be inclined downward toward the suctioning hole 160 from the discharging hole 150. The suctioning hole 160 is connected to a suction tube provided in the handpiece body H to generate suction power, thereby suctioning dead skin cells and fluid together to discharge to the outside.

In order to facilitate movement of the fluid, a downward inclined angle of the surface 120 is preferable to be 1 to 3 degrees.

### Second Exemplary Embodiment

Meanwhile, as shown in FIG. 5, a handpiece tip A2 according to the second exemplary embodiment is provided with a protruding part 140 having a concentric circular shape.

The protruding part 140 is composed of a plurality of walls 145 protruding in a spiral shape.

The wall 145 having the spiral shape is provided to have a lower height than that of the guide wall 180.

In addition, a flow path 144 through which the fluid flows is provided between the plurality of walls 145 protruding in the spiral shape.

Alternatively, the protruding part 140 may include a plurality of walls (not shown) protruding in the concentric circular shape. The wall having the concentric circular shape is provided to have a lower height than that of the guide wall 180.

A flow path 144 through which the fluid flows is provided between the plurality of walls having the concentric circular shape.

The above-described protruding part 140 is provided to have a curved part so that the corner portion is rounded to prevent skin damage.

### Third Exemplary Embodiment

As shown in FIG. 6, a handpiece tip A3 according to the third exemplary embodiment includes a contact part 100 to contact the skin, and a coupling part 200 having a space formed therein and coupled to the handpiece body.

The contact part 100 includes: a surface 120; a protruding part 140 provided to protrude on the outer side of the surface 120 and touching the skin; a discharging hole 150 formed on the surface 120 to discharge a therapeutic liquid; and a suctioning hole 160 formed on the surface 120 so as to suction a mixture of dead skin cells and the liquid, wherein the protruding part 140 includes a plurality of domes 147 configured to protrude in a hemispherical shape on the surface 120.

The plurality of domes 147 is provided by disposing in the circumferential direction on the outer circumferential edge of the discharging hole 150.

Preferably, the surface 120 is configured to protrude to be the hemispherical shape with the discharging hole 150 as the peak.

In addition, the surface 120 is provided to be inclined in one direction.

Therefore, the surface 120 protrudingly provided may allow the liquid discharged from the discharging hole 150 to flow to the edge of the surface 120, and then make the liquid be suctioned into the suctioning hole 160 formed at the edge thereof to be discharged therefrom.

Although the present disclosure has been described in association with the preferred embodiments, it will be readily apparent to those skilled in the art that various modifications and variations may be made without departing from the spirit and scope of the disclosure, and it is obvious that all such modifications and variations belong to the scope of the claims being attached.

What is claimed is:

1. A handpiece tip comprising:
a contact part to contact the skin; and
a coupling part having a space formed therein and coupled to a handpiece body,
wherein the contact part includes a surface, a protruding part provided to protrude on an outer side of the surface and touching the skin, a discharging hole formed on the surface to discharge a therapeutic liquid, and a suctioning hole formed on the surface so as to suction a mixture of dead skin cells and the liquid.

2. The handpiece tip of claim 1, wherein the protruding part is provided with a curved part to have a rounded corner portion, thereby preventing skin damage.

3. The handpiece tip of claim 1, further comprising:
a guide wall protrudingly provided on the outer side of the surface of the contact part; and
a plurality of partition walls connected to an inner circumferential surface of the guide wall and provided on the outer side of the surface of the contact part.

4. The handpiece tip of claim 1, further comprising:
a plurality of bits provided on the surface of the contact part and protrudingly provided in a circumferential direction around the discharging hole; and
a flow path provided between the plurality of bits to allow the fluid discharged from the discharging hole to flow.

5. The handpiece tip of claim 4, wherein
the flow path is provided with a flow path bottom on the surface of the contact part, and
the flow path bottom is configured as a horizontal bottom or as an inclined bottom inclined toward the suctioning hole from the discharging hole.

6. The handpiece tip of claim 1, wherein
the discharging hole is formed on a central part of the surface of the contact part, and
the suctioning hole is formed at an outer circumferential edge of the surface of the contact part.

7. The handpiece tip of claim 1, wherein the surface of the contact part is configured to be inclined downward toward the suctioning hole from the discharging hole.

8. The handpiece tip of claim 1, wherein the surface of the contact part is configured to protrude in a hemispherical shape with the discharging hole as a peak.
